(19) United States
(12) Patent Application Publication
(10) Pub. No.: US 2003/0088069 A1
(43) Pub. Date: May 8, 2003

(54) MUC1 isolation from bovine milk and whey

(76) Inventors: Sharyn X. Su, Plymouth, MN (US); Fang Ming, Madison Lake, MN (US); Mengyan Yang, Le Sueur, MN (US); Akimoto Ichinomiya, Tokushima (JP); Martin E. Davis, Tonka Bay, MN (US)

Correspondence Address: Schwegman, Lundberg, Woessner & Kluth, P.A., P.O. Box 2938, Minneapolis, MN, 55402, US

(21) Appl. No.: 10/223,112

(22) Filed: Aug. 15, 2002

Related U.S. Application Data
(63) Continuation of application No. PCT/US01/47359, filed on Nov. 7, 2001.

(51) Int. Cl. C07K 14/47

(52) U.S. Cl. 530/350

(57) ABSTRACT

A Process is provided for preparing MUC1 from bovine milk, whey or deproteinized whey. In one aspect the process involves providing concentrated whey derived from bovine milk; acidifying the whey; separating soluble whey protein from the resulting acidified whey to leave a MUC1-containing fraction containing insoluble membrane protein and low pI protein; and then separating MUC1 from the MUC1-containing fraction. In another, aspect the invention involves treating deproteinized whey derived from bovine milk with a whey protein solubilizing solution; centrifuging the treated whey to recover a soluble supernatant fraction; adsorbing protein on a buffer-washed PNA; separating the PNA from remaining liquid; and eluting MUC1 from said PNA. The MUC1 is pure and can be freeze dried.

### BACKGROUND OF THE INVENTION

This invention relates to methods or procedures to process and isolate MUC1 from bovine milk, whey or deproteinized whey.

MUC1, the epithelial mucin, is a high molecular weight glycoprotein and an integral membrane component. In milk, it is associated with milk fat-globule membrane (MFGM) that secreted from lactating cells in mammary gland. In bovine milk, the mucin is not very strongly held by the fat globules and can be easily released into milk serum by the variables such as cooling (or freezing), agitation, and age of the sample (Peterson, et al., 1998).

MUC1 genes from several mammals including human, bovine, mouse, were cloned or partially cloned (Spicer, et al., 1995). Although MUC1 mucins from different mammal species have relatively low homology in amino acid sequences, they are structurally related (Mather, 2000). The core protein consists of a number of distinct regions. It has an N-terminal signal sequence, a potentially highly glycosylated 20-amino acid repeats in the exoplasmic domain, the transmembrane anchor, and a plasmic tail. The high degree of polymorphism of the protein (there is often more than one polypeptide band shown on electrophoresis gel) was shown to be due to different numbers of tandem repeats present in the core protein. Serine, threonine, proline, alanine, and glycine account for about 60% of the amino acids. A complete bovine MUC1 sequence hasn't been reported however its tandem repeats, N-terminal 72 amino acids, and C-terminal 192 amino acids have been revealed.

About 50% of MUC1 are carbohydrates, and glycans may be linked to as much as one-third of the amino acids in certain regions of the mucin molecule. Most linkages are through hydroxyls of serine and threonine (O-linked), while some are through N-glycosylation via amide group of asparigines (Patton et al., 1995). This dense packing of the glycan in most parts of the molecule may prevent proteolytic degradation of the protein backbone and play an important role on the biochemical and functional characteristics of the mucin. The glycans vary greatly in length and may contain 1-20 residues.

Bovine MUC1 was earlier purified by two groups of scientists from MFGM. Cawston, et al. (1976) purified a glycoprotein A (later designated as MUC1) from bovine milk-fat globule membrane proteins by Sepharose 6B and hydroxylapatite column chromatography. Amino acid composition analysis showed that Serine, Proline, Threonine, Aspartic acid, and glycine are the major components. Another group (Snow et al., 1977) purified the major periodate-Schiff positive component (glycoprtein-2) by extraction of washed cream with chloroform/methanol followed by repeated chromatography on Sephadex G-200 in SDS (sodium dodecylsulphate) from bovine milk-fat globule membranes. The glycoprotein was >95% pure by SDS-PAGE (sodium dodecylsulphate—polyacrylamide gel electrophoresis). The molecular weight was 185,000 on 5% gel but changed to 70,000 on 12.5% gel. The glycoprotein contains 50% carbohydrate by weight, with sialic acid (30.5%), N-acetylglucosamine (22.3%), galactose (15.9%), N-acetylgalactosamine (14.0%), mannose (11.1%), and fucose (5.8%) being the major monosaccharides. Serine, glutamic acid, glycine, alanine, and threonine are the major amino acids. These proteins were later designated as bovine MUC1 (Patton et al., 1995).

Genetic polymorphism of MUC1 in bovine was well documented (Patton, et al., 1989; 1990; 1992; Huott, et al., 1995). Gel electrophoresis of bovine MFGM proteins can identify MUC1 bands easily with PAS (Protein A-Sepharose) or silver-staining. Because of genetic polymorphism, MUC1 may appear as a diffused band or as one or two discrete bands from individual animals. Five MUC1 bands ranging in apparent Mᵣ from approximately 156,000 to 193,000 have been identified from 119 Holstein cows. In the pooled milk samples of 800 commercial Holstein herds, MUC1 forms of 189,000, 177,000 and 156,000 are most common with the 156,000 form somewhat weaker (Huott, et al., 1995).

Mucin has been reported to function as physical barrier molecules to microorganisms and as lubricant on the surface of cells. The widely dispersed expression of mucin on cell surfaces coupled with the unique long filamentous physical properties implies protection of the cell. Human MUC1 has been shown to inhibit the adhesion of S-fimbriated E. coli to buccal epithelial cells (Schroten, et al., 1992). Purified human MUC1 was also shown to bind rotavirus and inhibit its infection (Peterson, et al., 1997).

Although human MUC1 has been studied extensively compared with other species, The limited resources make it difficult to commercialize its product. Since millions of pounds of milk are available for processing commercially everyday, it can be a good source for mucin production. While MUC1 in freshly prepared cow milk is associated with MFGM in the cream fraction, it is found mostly in the skim milk after cooling process (Peterson, et al. 1998). There is no published data on bovine MUC1 purification from skim milk or whey fractions and no purified MUC1 protein commercially available. Our objective is to develop processing methods for MUC1 isolation and purification from commercial sources of cow milk, whey or whey products with high efficiency and quality. In this disclosure, we are revealing our inventions of MUC1 isolation procedures using skim milk or whey.

Identification of Bovine MUC1

Possible MUC1 bands were identified by PAS-staining method which is specific for carbohydrates. Because MUC1 is the glycoprotein with the highest molecular weight among milk proteins and contains about 50% carbohydrates, MUC1 bands are easily identified by PAS staining and hardly can be stained with Coomassie Blue staining. Based on previous report on MUC1 bands shown on SDS-gel from pooled cows' milk, we identified possible MUC1 bands in concentrated whey and deproteinized whey with molecular weights between 160 to 190 Kd on SDS gel (FIG. 1). The band with slower electrophoresis mobility (around 180-190 kd) was stronger and more diffused, the one with faster mobility was weaker.

To confirm the mucin bands, C-terminal 16 amino acids peptide specific to bovine MUC1 was synthesized, conjugated and antiserum produced from rabbits. The antibody did not work with immuno-blotting but worked for immunoprecipitation. The high molecular weight bands was confirmed to be MUC1 by immunoprecipitation (IP) (FIG. 2). Both of the slow and the fast mobility mucin bands were precipitated by the peptide antibody. Identification of bovine MUC1

Possible MUC1 bands were identified by PAS-staining method which is specific for carbohydrates. Because MUC1 is the glycoprotein with the highest molecular weight among milk proteins and contains about 50% carbohydrates, MUC1 bands are easily identified by PAS staining and hardly can be stained with Coomassie Blue staining. Based on previous report on MUC1 bands shown on SDS-gel from pooled cows' milk, we identified possible MUC1 bands in concentrated whey and deproteinized whey with molecular weights between 160 to 190 Kd on SDS gel (FIG. 1). The band with slower electrophoresis mobility (around 180-190 kd) was stronger and more diffused, the one with faster mobility was weaker.

To confirm the mucin bands, C-terminal 16 amino acids peptide specific to bovine MUC1 was synthesized, conjugated and antiserum produced from rabbits. The antibody did not work with immuno-blotting but worked for immunoprecipitation. The high molecular weight bands was confirmed to be MUC1 by immunoprecipitation (IP) (FIG. 2). Both of the slow and the fast mobility mucin bands were precipitated by the peptide antibody.

### BRIEF DESCRIPTION OF THE DRAWINGS

The invention will be better understood and its advantages more apparent from the following description, especially when read in light of the accompanying drawings, wherein:

FIG. 1 is an SDS-PAGE of concentrated whey and DPW (deproteinized whey) showing possible MUC1 bands in concentrated whey and deproteinized whey with molecular weights between 160 to 190 Kd on SDS gel.

FIG. 2 is a 4-15% SDS-ME PAGE, immunoprecipitation result using MUC1 specific peptide antibody, PAS-CBB-Silver stained, confirmed high molecular weight bands to be MUC1.

FIG. 3 is a 7.5% SDS-ME PAGE, PAS and Coomassie Blue stained.

### SUMMARY OF THE INVENTION

It is an object of the invention to provide an improved method for preparing MUC1.

These and other objects are accomplished by the invention, which provides an improved method for preparing MUC1 an improved method for preparing MUC1, which is described in a preferred form below and is illustrated in the attached drawings and the following Examples.

In one aspect the invention provides a process for preparing MUC1 from bovine milk, whey or deproteinized whey as described in the examples below.

In another aspect, the invention provides a process for preparing MUC1 from bovine milk, comprising:

(a) providing whey derived from bovine milk;

(b) concentrating said whey;

(c) acidifying said whey to a pH below 4.0;

(d) separating soluble whey protein from the resulting acidified whey to leave a MUC1-(e)

(e) containing fraction containing insoluble membrane protein and low pI protein; and

(f) separating MUC1 from the MUC1-containing fraction.

In yet another aspect, the invention provides a process for preparing MUC1 from bovine milk, comprising:

(a) treating deproteinized whey derived from bovine milk with a whey protein solubilizing solution;

(b) centrifuging the whey treated in step (a) to recover a soluble supernatant fraction;

(c) adsorbing protein on a buffer-washed PNA;

(d) separating said PNA from remaining liquid; and

(e) eluting MUC1 from said PNA.

Preferred forms are illustrated in the examples.

### DETAILED DESCRIPTION OF THE INVENTION

Isolation of MUC1

Cooled milk from dairy farms was de-fatted by centrifugation. The skim milk was adjusted to pH4.6 and casein was precipitated out at this pH, producing whey, which was used for mucin purification. Bovine whey provided by Le Sueur cheese plant was also used as starting material for mucin purification.

The whey was concentrated about 3 fold by UF membrane and reacted with Sephadex SP ion exchange resin at pH3.5 to adsorb the major whey proteins. The residue was concentrated with MF membranes and diafiltrated in water. The concentrated, deproteinized whey (DPW) was diluted to about 2-3% of total protein in 1-3% Triton X-100 with TBS (pH8) or 10 mM HEPES (pH7.5) plus 0.5M NaCl. The solution was centrifuged and supernatant recovered by removing the top fat layer. Several different kinds of lectins and the peptide antibody were tried for mucin binding test. Among the tested, peanut agglutinin (PNA) binds to the mucin most specifically and abundantly. PNA-agarose affinity chromatography was performed as a final step of MUC1 purification. After elute with 10% lactose; the eluate was concentrated and diafiltrated in distilled water by UF membranes and freeze-dried.

The following Examples are provided to further illustrate and explain a preferred form of the invention and are not to be taken as limiting in any regard. Unless otherwise indicated, all parts and percentages are by weight.

### EXAMPLE 1

### Source of Bovine Milk

Davisco Cheese plant at Le Sueur provided bovine milk that was purchased from dairy farmers.

### EXAMPLE 2

### Bovine Whey Preparation

Bovine whey was prepared according to Kanamaru et al. (1993) or directly from Le Sueur cheese plant. Briefly, bovine milk was centrifuged to remove the top fat layer and to collect the skim milk. Bovine whey was obtained by adjusting pH to 4.6 and removing casein precipitate by centrifugation.

### EXAMPLE 3

### Preparation of MF Concentrated Deproteinized Whey (DPW)

Bovine whey was concentrated three folds by ultrafiltration membrane and the concentrated whey was adjusted to pH around 3.5. The whey with low pH was used for SP ion-exchange chromatography to remove the major whey proteins. The residue that contains mostly insoluble membrane proteins and proteins with low pI (isoelectric point) values was diafiltrated and concentrated to about 10-15% of total protein via microfiltration membrane.

### EXAMPLE 4

### MUC1 Identification from Whey

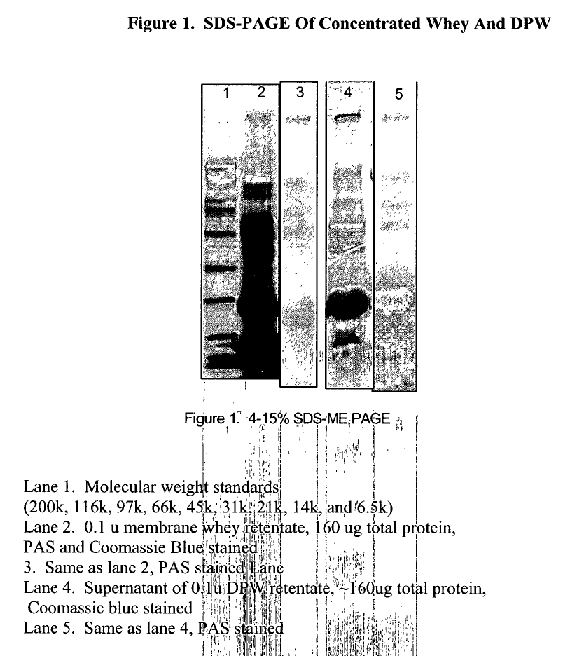

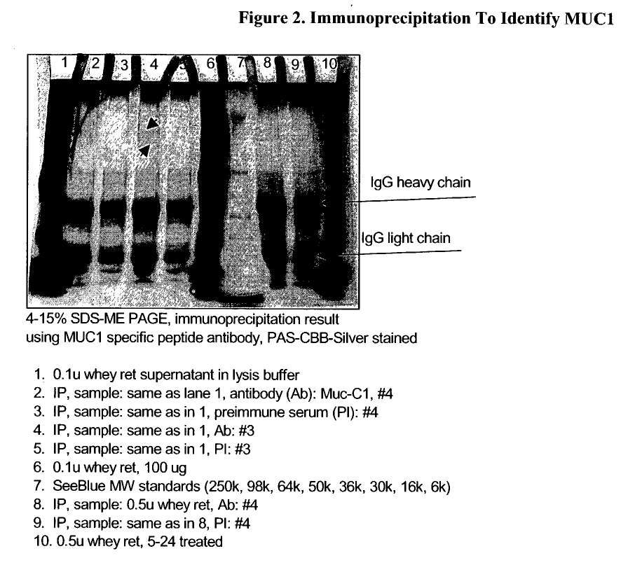

4-15% acrylamide gel was used to run MF membrane concentrated whey and DPW protein and PAS-staining was used to stain the glycoproteins followed by Coomassie blue staining to stain total proteins (FIG. 1). Possible MUC1 was identified as two diffused PAS bands of molecular weights around 180-190 kd and 160 kd, with a stronger staining for the higher molecular band, and these bands can not be stained with Coomassie blue as reported. C-terminal amino acid sequence of bovine MUC1 was published (Spicer et al., 1995) and shown to be best conserved among all the mammalian species tested. C-terminal 16 amino acids peptide specific for bovine MUC1 was synthesized by Genemed Synthesis, Inc. and antiserum produced from rabbits by the same company. Immunoprecipitation was performed mixing the antiserum and the concentrated whey and the result was shown on a SDS-gel (FIG. 2). The Immunoprecipitation results confirmed that the two highest molecular weight glycoprotein bands were MUC1.

### EXAMPLE 5

### Purification of MUC1 by Peanut Agglutinin (PNA)-Resin Affinity Chromatography

Concentrated and diafiltrated DPW was diluted to about 2-3% total protein concentration with buffer A (2-3% Triton X-100, 0.5M NaCl, 50 mM Tris-HCl, pH8), solubilizing for 1 hour at room temperature with constant stirring. The solubilized mixture was centrifuged at 21,000×g for 3040 minutes at 4-6° C. The top fat layer was removed and the center supernatant layer pooled. The agarose immobilized PNA was purchased from Vector laboratories and washed with several volumes of buffer B (1% Triton X-100, 0.1 mM CaCl₂, in TBS, pH8). The buffer B washed PNA-resin was mixed with the solubilized protein mixture and let interact at room temperature for 30-60 minutes by stirring in a reactor. The unbound residue was pumped out and the resin was washed 2-3 times with buffer B followed by TBS buffer until the absorbency at 280 nm was close to zero. MUC1 was eluted by stirring in 1-2 volumes of elution buffer (10% lactose or 200 mM galactose in TBS) for 20 minute at room temperature each time, repeated for two more times. The eluate was pooled, concentrated, and diafiltrated by Spectrum ultrafiltration membrane (50K) and in a stirred ultrafiltration cell (30 k, from Millipore) with distilled water. The concentrated and diafiltrated Muc1 may be freeze dried.

### EXAMPLE 6

### Purity of the Purified MUC1

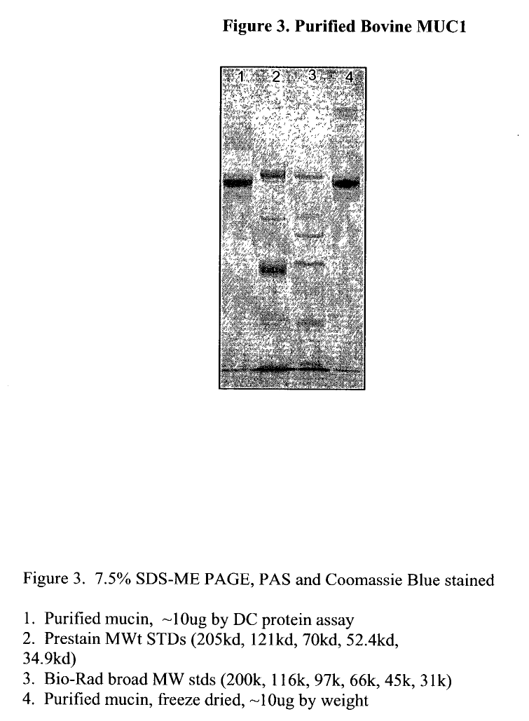

The purified MUC1 was subjected to 7.5% or 4-15% SDS-PAGE to check for purity. FIG. 3 showed a 7.5% gel, loaded with about 10 ug of MUC1 each lane, was stained with PAS followed by Commassie blue. Two major glycoprotein bands were shown in the molecular weight range of 180-190 kd and 160 kd respectively and three very faint bands with molecular weights range from 60 to 78 kd. Scanning the gel at 560 nm wavelength with Beckman spectrophotometer (DU 650) showed that the MUC1 bands comprised over 90%-95% of the total protein.

### EXAMPLE 7

### Amino Acid Composition and Sialic Acid Analysis for the Purified MUC1

Amino acid composition analysis of the purified bovine MUC1 was performed at Protein Chemistry Laboratory of University of Texas Medical Branch. The protein was rich in serine, proline and threonine, which is consistent with reported for MUC1. Table 1 shows a comparison of amino acid compositions of our purified bovine MUC1 with that of other two labs (Cawston, et al., 1976 and Snow, et al., 1977). Sialic acid was analyzed using modified TBA assay after enzymatic releasing. About 16% of sialic acid in MUC1 by weight were obtained, which was close to that reported by Snow et al. (13%).

TABLE 1
Comparison Of Amino Acid Compositions Of
Bovine MUC1 From 3 Sources
(Values Are Expressed As Mole Percentage Of Amino Acids Recovered)

| | Cawston et al. | Snow et al. | Davisco |
|---|---|---|---|
| Amino acid | (1976) | (1977) | Foods |
| Ser | 13.6 | 15.6 | 13.6 |
| Pro | 11.0 | 7.2 | 11.2 |
| Thr | 9.7 | 8.5 | 10.3 |
| Ala | 6.8 | 9.2 | 9.4 |
| Glu | 7.5 | 12.6 | 7.8 |
| Leu | 6.3 | 6.0 | 7.1 |
| Gly | 8.0 | 11.2 | 6.7 |
| Asp | 8.3 | 6.3 | 6.4 |
| Val | 2.9 | 3.9 | 5.7 |
| Ile | 2.9 | 3.1 | 4.0 |
| Arg | 2.8 | 3.7 | 3.8 |
| Lys | 3.3 | 4.3 | 3.5 |
| Met | 1.1 | 0.83 | 2.8 |
| Phe | 8.0 | 2.5 | 2.7 |
| His | 2.2 | 3.6 | 2.7 |
| Tyr | 4.8 | 1.4 | 2.1 |

### EXAMPLE 8

### Some Physical Properties Of MUC1

The freeze-dried MUC1 had white color. When dissolved in distilled water at a concentration of 7-10%, it looked light brownish-yellow and very viscous. Viscosity test of 5% (w/w) purified MUC1 by Zeiffuchs Cross-Ann Viscometer revealed a viscosity of 30.4 cSt, while 10% (w/w) BiPro solution (which is highly purified whey proteins produced at Davisco Foods) had a viscosity of only 1.72 cSt.

The above description is intended to enable the person skilled in the art to practice the invention. It is not intended to detail all of the possible modifications and variations which will become apparent to the skilled worker upon reading the description. It is intended, however, that all such modifications and variations be included within the scope of the invention which is seen in the above description and otherwise defined by the following claims. The claims are meant to cover the indicated elements and steps in any arrangement or sequence which is effective to meet the objectives intended for the invention, unless the context specifically indicates the contrary.

### Publications

1. Cawston, T. E., M. Anderson, and G. C. Cheeseman. 1976. Isolation, Preparation and the Amino Acid Composition of 4 Milk-fat Globule Membrane Protein Solubilized by Treatment with Sodium Dodecyl Sulphate. J. Dairy Research 43: 401-409.

2. Huott, M. L., R. V. Josephson, J. R. Hens, G. W. Rogers, and S. Patton. 1995. Polymorphic Forms of the Epithelial Mucin, PAS-I (MUC1), in Milk of Holstein Cows (Bos Taurus). Comp. Biochem. Physiol. 111B: 559-565.

3. Kanamaru, Y., T. Toyoki, S. Nagaoka, Y. Kuzuya, and R. Niki. 1993. High Molecular Weight Mucin-like Glycoprotein in Bovine Milk. Biosci. Biotech. Biochem. 57(4): 666-667.

4. Mather, I. H. 2000. A Review and Proposed Nomenclature for Major Proteins of the Milk-Fat Globule Membrane. J. Dairy Sci 83:203-247.

5. Patton, S., and L. D. Muller. 1991. Genetic Polymorphism of the Epithelial Mucin, PAS-I, in Milk Samples from the Major Dairy Breeds. J. Dairy Sci. 75:863-867.

6. Patton, S., and R. S. Patton. 1990. Genetic Polymorphism of PAS-I, the Mucin-like Glycoprotein of Bovine Milk Fat Globule Membrane. J. Dairy Sci. 73:3567-3574.

7. Patton, S., G. E. Huston, R. Jenness, and Y, Vaucher. 1989. Differences Between Individuals in High-molecular Weight Glycoprotein from Mammary Epithelia of Several Species. Biochemica et Biophysica Acta 980:333-338.

8. Patton, S., S. J. Gendler, and A. P. Spicer. 1995. The Epithelial Mucin, MUC1, of Milk, Mammary Grand and Other Tissues. Biochemica et Biophysica Acta 1241:407-424.

9. Peterson, J. A., S. Patton, M. Hamosh. 1998. Glycoproteins of the Human Milk Fat Globule in the Protection of the Breast-Fed Infant Against Infections. Biol Neonate 74:143-162.

10. Schroten, H., F. G. Hanisch, R. Plogmann, J. Hacker, G. Uhlenbruck, R. Nobis-Bosch, and V. Wahn. 1992. Inhibition of Adhesion of S-Fimbriated Escherichia Coli to Buccal Epithelial Cells by Human Milk Fat Globule Membrane Components: a Novel Aspect of the Protective Function of Mucins in the Nonimmunoglobulin Fraction. Infection And Immunity 60:2893-2899.

11. Snow, L. D., D. G. Colton, and K. L. Carraway. 1977. Purification and Properties of the Major Sialoglycoprotein of the Milk Fat Globule Membrane. Archives of Biochemistry And Biophysics 179:690-697.

12. Spicer, A. P., T. Duhig, B. S. Chilton, and S. J. Gendler. 1995. Analysis of Mammalian MUC1 Genes Reveals Potential Functionally Important Domains. Mammalian Genome 6:885

13. Patent: Peterson, et al. 1997. Anti-viral composition and kit and use for treating potavirus infection and diarrhea. U.S. Pat. NO. 5,667,797

1. A process for preparing MUC1 from bovine milk, whey or deproteinized whey as described in the examples above.

2. A process for preparing MUC1 from bovine milk, comprising:
(a) providing whey derived from bovine milk;
(b) concentrating said whey;
(c) acidifying said whey to a pH below 4.0;
(d) separating soluble whey protein from the resulting acidified whey to leave a MUC1-(e)
(e) containing fraction containing insoluble membrane protein and low pI protein; and
(f) separating MUC1 from the MUC1-containing fraction.

3. A process for preparing MUC1 from bovine milk, comprising:
(a) treating deproteinized whey derived from bovine milk with a whey protein solubilizing solution
(b) centrifuging the whey treated in step (a) to recover a soluble supernatant fraction;
(c) adsorbing protein on a buffer-washed PNA;
(d) separating said PNA from remaining liquid; and
(e) eluting MUC1 from said PNA.

4. MUC1 prepared according to any of claims 1 to 3.
